(19) United States
(12) Patent Application Publication
(10) Pub. No.: US 2002/0160368 A1
(43) Pub. Date: Oct. 31, 2002

(54) Chemical arrays with oriented rows

(76) Inventors: Jay K.. Bass, Mountain View, CA (US); John F. McEntee, Boulder Creek, CA (US); Tim J. Lazaruk, Redwood City, CA (US); Maryam Mobed-Miremadi, Sunnyvale, CA (US); Brent T. Tolosko, Santa Clara, CA (US)

Correspondence Address: AGILENT TECHNOLOGIES, INC., Legal Department, 51U-PD, Intellectual Property Administration, P.O. Box 58043, Santa Clara, CA, 95052-8043, US

(21) Appl. No.: 09/846,058

(22) Filed: Apr. 30, 2001

(51) Int. Cl. C12Q 1/68; B05D 3/00; G01N 33/53; G01N 33/542; C12M 1/34

(52) U.S. Cl. 435/6; 435/7.9; 435/287.2; 427/2.11

(57) ABSTRACT

A method of fabricating an array of multiple features of different chemical moieties on a substrate surface, and a method of using arrays so fabricated. The fabrication method includes determining an identity of a first direction across the substrate surface (such as a drawn direction) along which the substrate surface has a higher height uniformity than along a second direction across the substrate. Chemical moieties are placed on the substrate so as to provide features thereon along rows more closely aligned with the first direction than the second direction.

### FIELD OF THE INVENTION

This invention relates to arrays, for example arrays of different chemicals such as biopolymer arrays (for example, polynucleotide arrays including DNA arrays) which can be used in diagnostic, screening, gene expression analysis, and other applications.

### BACKGROUND OF THE INVENTION

Arrays of biopolymers, such as arrays of peptides or polynucleotides (such as DNA or RNA), are known and are used, for example, as diagnostic or screening tools. Such arrays include regions (sometimes referenced as features or spots) of usually different sequence biopolymers arranged in a predetermined configuration on a substrate. The arrays, when exposed to a sample, will exhibit a pattern of binding which is indicative of the presence and/or concentration of one or more components of the sample, such as an antigen in the case of a peptide array or a polynucleotide of particular sequence in the case of a polynucleotide array. The binding pattern can be detected by reading the array, for example, by observing a fluorescence pattern on the array following exposure to a fluid sample in which all potential targets (for example, DNA) in the sample have been labeled with a suitable fluorescent label.

Methods of fabricating biopolymer arrays can be fabricated using light directed methods, in situ synthesis methods or deposition of the previously obtained biopolymers. In known light directed synthesis methods the aim is to form an array of oligonucleotides on a surface by removing a photoremovable group from a surface, coupling a monomer to the exposed region of the surface, and repeating the process. The in situ synthesis methods include those described in U.S. Pat. No. 5,449,754 for synthesizing peptide arrays, as well as U.S. Pat. No. 6,180,351 and WO 98/41531 and the references cited in them for synthesizing polynucleotides (specifically, DNA). Such in situ synthesis methods can be basically regarded as iterating the sequence of depositing droplets of: (a) a protected monomer onto predetermined locations on a substrate to link with either a suitably activated substrate surface (or with a previously deposited deprotected monomer); (b) deprotecting the deposited monomer so that it can now react with a subsequently deposited protected monomer; and (c) depositing another protected monomer for linking. Different monomers may be deposited at different regions on the substrate during any one iteration so that the different regions of the completed array will have different desired biopolymer sequences. One or more intermediate further steps may be required in each iteration, such as oxidation and washing steps.

The “deposition method” basically involve depositing previously obtained biopolymers at predetermined locations on a substrate which are suitably activated such that the biopolymers can link thereto. The deposited biopolymers may, for example, be obtained from synthetic or biological sources. Biopolymers of different sequence may be deposited at different regions of the substrate to yield the completed array. Washing or other additional steps may also be used. Typical procedures known in the art for deposition of polynucleotides, particularly DNA such as whole oligomers or cDNA, are to load a small volume of DNA in solution in one or more drop dispensers such as the tip of a pin or in an open capillary and, touch the pin or capillary to the surface of the substrate. Such a procedure is described in U.S. Pat. No. 5,807,522. When the fluid touches the surface, some of the fluid is transferred. The pin or capillary must be washed prior to picking up the next type of DNA for spotting onto the array. This process is repeated for many different sequences and, eventually, the desired array is formed. Alternatively, the DNA can be loaded into a drop dispenser in the form of a pulse jet, such as an inkjet head, and fired onto the substrate. Such a technique has been described, for example, in U.S. Pat. No. 6,180,351, PCT publications WO 95/25116 and WO 98/41531, and elsewhere. This method has the advantage of non-contact deposition. Still other methods include pipetting and positive displacement pumps such as the Biodot equipment (available from Bio-Dot Inc., Irvine Calif., USA).

In array fabrication, the quantities of DNA available for the array are usually very small and expensive. Sample quantities available for testing are usually also very small and it is therefore desirable to simultaneously test the same sample against a large number of different probes on an array. These conditions favor the use of arrays with large numbers of very small, closely spaced spots (features). However, it is important that the features be equal sized and that different features on an exposed array can be read with equal accuracy. Differences in these quantities can lead to errors in interpretation of results from a read array with potentially serious negative implications for an experiment or diagnosis.

### SUMMARY OF THE INVENTION

The present invention realizes that a surface of a substrate on which arrays are fabricated, are not perfectly flat. That is, there are relative variations in height moving across the surface (high variations in height being referenced as low height uniformity). As a result, when an array is being fabricated the distance between a pulse jet orifice from which a drop is fired and the substrate surface will vary. Consequently, this may lead to variations in the size or shape of a deposited drop. Furthermore, when a fabricated array is read such as by a scanner, the optical path length to or from features on the substrate surface may vary as a result of such substrate surface variations. This may lead to variations in the detection of features as they are read. While a scanner can be equipped with an autofocus system to compensate for substrate surface height variations to some extent, typical autofocus systems will require some time to react to a change in substrate surface height variations. Given that the features are small (for example, down to 20 or 10 μm) there is little time for an autofocus system to adjust to substrate surface height variations that occur over the distance of one or several features, particularly where it is desired to use higher scanning speeds. This can lead to multiple signal variations as a scanner scans along a given row of hundreds of features.

The present invention has further realized though, that many types of substrates may be manufactured by a drawing operation. For example, drawn glass substrates is manufactured by a well known process where molten glass is pulled through an elongated thin slot to form a long ribbon of glass extending lengthwise in the drawn direction and with a width approximately equal to the length of the slot. As a result of this process, conditions at any one position on the glass ribbon width (that is, a line parallel to the drawn direction) will remain relatively constant along the length of the glass ribbon, while conditions at multiple different positions along the glass ribbon width (that is, across the drawn direction) will tend to have vary more. As a result, the present invention realizes that the most significant variations in a substrate surface height for a drawn substrate, are likely to occur in a direction lying across (and typically orthogonal to) the drawn direction.

The present invention then, provides in one aspect a method of fabricating an array of multiple features of different chemical moieties (such as biopolymers) on a surface of a substrate. The method includes determining an identity of a first direction across the substrate surface along which the substrate surface has a higher height uniformity than along a second direction across the substrate. The chemical moieties are placed on the substrate so as to provide features thereon along rows more closely aligned with the first direction (for example, parallel with the first direction) than the second direction.

In another aspect of the invention, a method of fabricating an array of multiple features of different chemical moieties on a surface of a drawn substrate, is provided. This method includes determining an identity of a drawn direction of the substrate, and placing the chemical moieties on the substrate surface so as to provide features thereon along linear rows oriented adjacent the drawn direction.

The determining in any aspect may, for example, be performed by measuring surface height variations such as by measuring the thickness of the substrate at a sufficient number of different positions (for example, in the case of a rectangular substrate, along two orthogonal directions on the surface), or by receiving in association with the substrate, an identification of the first or drawn direction (for example, receiving this from a remote location which may or may not be the same as a remote location from which the substrate was received). The identification may be received directly, such as with a written description forwarded from the remote location which is packaged or otherwise associated with the substrate. The identification may also be received indirectly, such as by retrieval from a memory (remote or local, such as a portable storage medium) in response to providing an identifier on the substrate.

The method may additionally include associating with the array, an identification as to the direction of the rows and forwarding the array and associated identification to a remote location. The forwarding may, for example, include applying an identifier (such as a fiducial mark or code) to the substrate or a housing for the substrate, and saving the identification in a memory in association with the identifier. The identification may, for example, be a reference to a shape characteristic of the substrate or a housing for the substrate (for example, where the substrate is rectangular with the first or drawn direction extending parallel with a longer dimension of the substrate, the identification may simply refer to this).

The method may include placing the chemical moieties on the substrate surface so as to provide features thereon along linear rows oriented parallel to the first or drawn direction, by depositing drops from a drop deposition head while moving the head along one of the rows parallel with the first or drawn direction. This can be repeated multiple times as necessary at another one of the rows parallel with the drawn direction, until the array is formed.

The present invention further provides a method of reading an array of multiple features of different chemical moieties on a substrate surface, the array having rows of features. Such a method may include determining an identity of a first direction across the substrate along which the substrate surface has a higher height uniformity than along a second direction across the substrate, or determining an a drawn direction, and repeatedly scanning an illuminating beam across features in parallel paths which are more closely aligned with the first direction than the second direction (or closely aligned, for example parallel with, the drawn direction). The determining can be performed by any of those same methods for determining described above (with the possibility of the reference to the substrate instead being a reference to the substrate, array, or a housing carrying the substrate and array).

One or more of the methods of the present invention may provide one or more of the following, or other, useful benefits. For example, during scanning of an array fabricated by a method of the present invention, a scanning beam can be scanned across features on a substrate surface, in a direction which in which there is likely to be less substrate surface variations. During fabrication where drops are deposited from a head moved over the substrate, the direction of movement can be selected so that there is less likely to be subs

### BRIEF DESCRIPTION OF THE DRAWINGS

Embodiments of the invention will now be described with reference to the drawings in which:

FIG. 1 illustrates a substrate carrying multiple arrays, such as may be fabricated by methods of the present invention;

FIG. 2 is an enlarged view of a portion of FIG. 1 showing multiple ideal spots or features;

FIG. 3 is an enlarged cross-section of a portion of the array in FIG. 2 along a second direction;

FIG. 4 is an enlarged cross-section of a portion of another array along the second direction;

FIG. 5 is an enlarged cross-section of a portion of the array of FIG. 4 but along the drawn direction;

FIG. 6 illustrates refractive power measurement (in millidiopters) of an actual piece of drawn glass as might be used as a substrate on which an array is fabricated by a method of the present invention (lines of equal color representing the same refractive power measurement);

FIG. 7 is a a measurement of refractive power (in millidiopters) versus width (in mm) taken across the glass of FIG. 6;

FIG. 8 is a schematic diagram of an apparatus which can fabricate arrays such as those of FIGS. 1-5, in accordance with a method of the present invention; and

FIG. 9 is a schematic diagram of an apparatus at a user site which can read an array in accordance with a method of the present invention.

To facilitate understanding, identical reference numerals have been used, where practical, to designate identical elements that are common to the figures.

### DETAILED DESCRIPTION OF THE INVENTION

In the present application, unless a contrary intention appears, the following terms refer to the indicated characteristics. A “biopolymer” is a polymer of one or more types of repeating units. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides.

An “array”, unless a contrary intention appears, includes any one, two or three dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces and present in an ordered pattern such as in linear rows and columns. In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). An “array layout” refers collectively to one or more physical, chemical or biological characteristics of the features, such as feature positioning, one or more feature dimensions, errors, or some indication of a moiety at a given location. “Hybridizing” and “binding”, with respect to polynucleotides, are used interchangeably.

When one item is indicated as being “remote” from another, this is referenced that the two items are at least in different buildings, and may be at least one mile, ten miles, or at least one hundred miles apart. “Communicating” information references transmitting the data representing that information as electric or electromagnetic (including light) signals over a suitable communication channel (for example, a private or public network). “Forwarding” an item refers to any means of getting that item from one location to the next, such as by causing the item to be physically transported (shipped) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports).

Height “uniformity” of a substrate surface, refers to variation in relative height of the surface moving across the surface in a predetermined direction. A surface with a number of parameters, including the orifice diameter, the orifice length (thickness of the orifice member at the orifice), the size of the deposition chamber, and the size of the heating element, among others. The amount of fluid that is expelled during a single activation event is generally in the range about 0.1 to 1000 pL, usually about 0.5 to 500 pL and more usually about 1.0 to 250 pL. A typical velocity at which the fluid is expelled from the chamber is more than about 1 m/s, usually more than about 10 m/s, and may be as great as about 20 m/s or greater. As will be appreciated, if the orifice is in motion with respect to the receiving surface at the time an ejector is activated, the actual site of deposition of the material will not be the location that is at the moment of activation in a line-of-sight relation to the orifice, but will be a location that is predictable for the given distances and velocities.

The apparatus can deposit droplets to provide features which may have widths (that is, diameter, for a round spot) in the range from a minimum of about 10 μm to a maximum of about 1.0 cm. In embodiments where very small spot sizes or feature sizes are desired, material can be deposited according to the invention in small spots whose width is in the range about 1.0 μm to 1.0 mm, usually about 5.0 μm to 500 μm, and more usually about 10 μm to 200 μm.

The apparatus further includes a display 310, speaker 314, and operator input device 312. Operator input device 312 may, for example, be a keyboard, mouse, or the like. Processor 140 has access to a memory 141, and controls print head 210 (specifically, the activation of the ejectors therein), operation of the positioning system, operation of each jet in print head 210, and operation of display 310 and speaker 314. Memory 141 may be any suitable device or devices in which processor 140 can store and retrieve data, such as magnetic, optical, or solid state storage devices (including magnetic or optical disks or tape or RAM, or any other suitable device, either fixed or portable). Processor 140 may include a general purpose digital microprocessor suitably programmed from a computer readable medium carrying necessary program code, to execute all of the steps required for by the present invention for array production, or any hardware or software combination which will perform those or equivalent steps. The programming can be provided remotely to processor 140, or previously saved in a computer program product such as memory 141 or some other portable or fixed computer readable storage medium using any of those devices mentioned below in connection with memory 141. For example, a magnetic or optical disk 324a may carry the programming, and can be read by disk writer/reader 326.

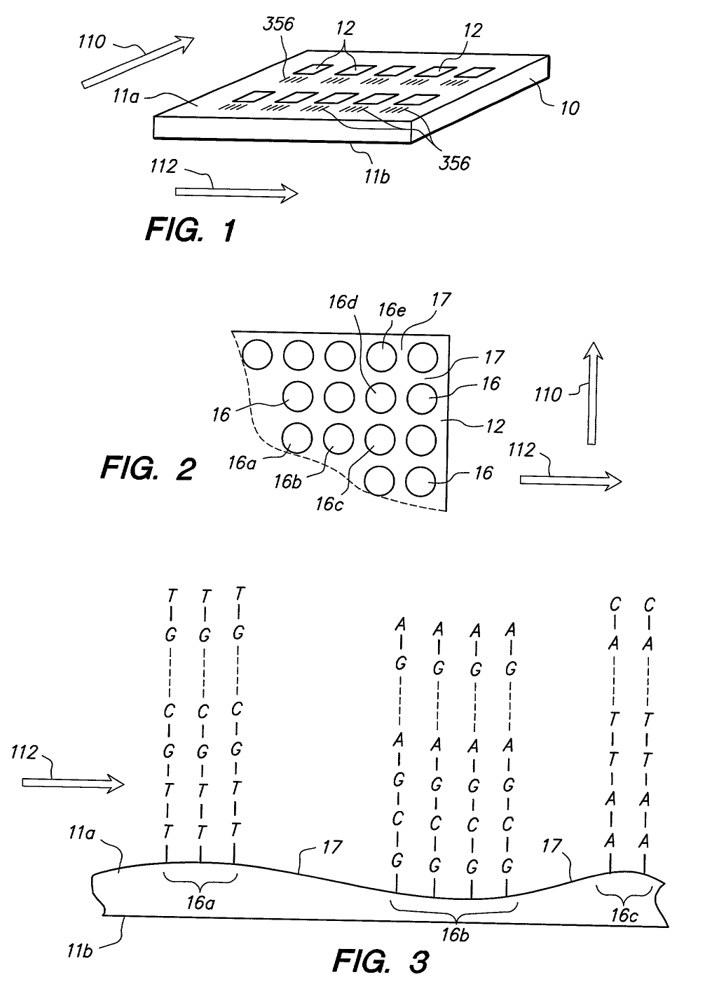

A writing system which is under the control of processor 140, includes a writer in the form of a printer 150 which applies identifiers onto substrate 10 by printing them in the form of the bar codes 356 directly onto substrate 10 (or indirectly such as onto a label later attached to the substrate), each in association with a corresponding array 12 as illustrated in FIG. 1. Alternatively, the identifiers can by applied onto a housing (not shown) carrying the substrate or to a label to be applied to such substrate or housing. Printer 150 may accomplish this task before or after formation of the array by the drop deposition system. The identifiers may include a communication address which can identify to a location (such as an end user station) an address of a location on communication channel 180 from which will be communicated from a remote location an identification of first direction 110 or second direction 112 for an array 12 in response to a received communication of the identifier for that array 12. Such identification may be indirect, for example simply as an instruction for the remote location to scan a beam along lines of features 16 on an array 12 in an identified direction (which is actually the first or drawn direction) or may be direct (in the sense that it explicitly identifies a direction as being the drawn direction). Optionally, bar code 356 may contain such identification itself. In the case of a communication of the identification information of the first or second direction from a remote location, additional data may also be communicated, such as, feature characteristic data which may include feature physical characteristics or biological function data for one or more of the biopolymers on array features, as described more fully in U.S. patent application Ser. No. 09/775387 titled “Chemical Array Fabrication And Use” by Cattell filed on Jan. 31, 2001. Such remote location will have a memory accessible on the communication channel 180 carrying a database of the data in association with corresponding array identifiers or corresponding biopolymer identity information so as to facilitate retrieval of the data upon receipt of the array identifier or biopolymer identity information. The location identified by the communication address may be that of communication module 144 or alternatively that of another location. Examples of a communication address may be a telephone number, computer ID on a WAN, or an internet Universal Resource Locator. The writing system further includes a data writer/reader 326 (such as an optical or magnetic disk drive) which can write data to a portable computer readable storage medium (such as an optical or magnetic disk). A cutter 152 is provided to cut substrate 10 into individual array units 15 each carrying a corresponding array 12 and bar code 356.

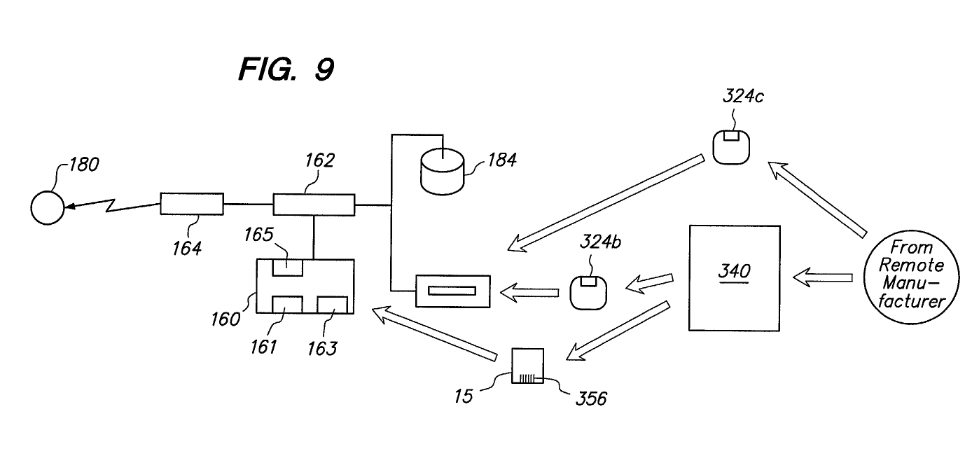

FIG. 9 illustrates an apparatus at which an addressable array 12 may be used, in particular a single “user station” (usually at the location of the customer which ordered a received array 12) which is remote from the fabrication station. The user station includes a processor 162, a memory 184, an array reader in the form of a scanner 160 to read an array following exposure to a sample, data writer/reader 186 (which may be capable of writing/reading to the same type of media as writer/reader 320), and a communication module 164 which also has access to communication channel 180. Scanner 160 may include a holder 161 which receives and holds an array unit 15, as well as a source of illumination (such as a laser) and a light sensor 165 to read fluorescent light signals from respective features on the array. Scanner 160 is typically constructed to scan a laser beam across an array 12 line by line in a known rectangular manner. That is, the beam is scanned across one line of features on an array 12 from a first end to a second end, then the beam is moved to a next adjacent line and that line is scanned from a second end to a first end, and the process repeated moving down one line at a time until the entire array 12 is scanned. Alternatively, a zigzag scan can be used in which after the beam is scanned from a first to second end of each line, it is moved to a next adjacent line and again scanned from a first to second end. Scanner 160 may include an autofocus system as more fully described, for example, in U.S. patent application Ser. No. 09/415184 titled “Apparatus And Method For Autofocus” by Dorsel et al. (filed Oct. 7, 1999). Communication module 164 may be any type of suitable communication module, such as those described in connection with communication module 144. Memory 184 can be any type of memory such as those used for memory 141. Scanner 160 can be any suitable apparatus for reading an array, such as one which can read the location and intensity of fluorescence at each feature of an array following exposure to a fluorescently labeled sample. For example, such a scanner may be similar to the GENEARRAY scanner available from Hewlett-Packard, Palo Alto, Calif. Scanner 160 also includes though, a reader 163 to read a the identifier in the form bar code 356 appearing on segment 15 as a read identifier signal. However, less preferably this reader may be the same as the array reader. The scanning components of scanner 160, holder 161, and reader 163 may all be contained within the same housing of a single same apparatus.

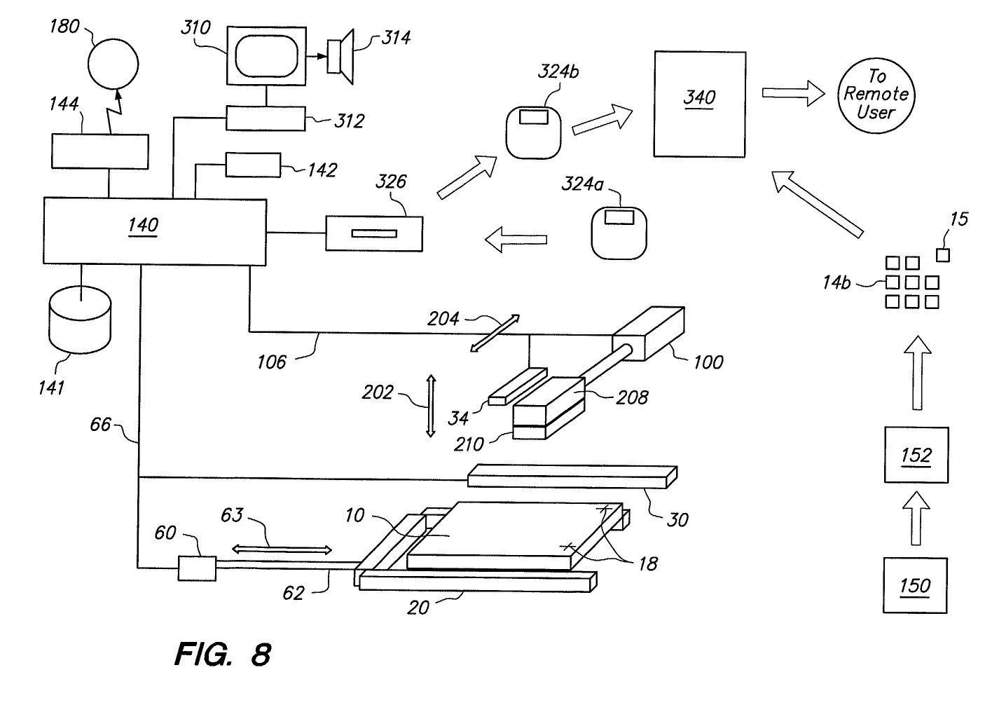

It will be understood that there may be multiple such user stations of FIG. 9, each remote from the fabrication station and each other, with the fabrication station of FIG. 8 acting as a central fabrication station (that is, a fabrication station which services more than one remote user station at the same or different times). One or more such user stations may be in communication with the fabrication station at any given time. It will also be appreciated that processors 140 and 162 can be programmed from any computer readable medium carrying a suitable computer program. For example, such a medium can be any memory device such as those described in connection with memory 141, and may be read locally (such as by reader/writer 320 in the case of processor 140 or writer/reader 186 in the case of processor 162) or from a remote location through communication channel 180.

The operation of the fabrication station will now be described with reference to FIGS. 8 and 9 in particular. Events for only one user station of FIG. 9 remote from the central fabrication station of FIG. 8 are described, but it will be understood that typically there will be many such remote user stations. The identity of drawn direction 110 of substrate 10 is first determined in any of the manners already described. As mentioned on a rectangular substrate 10 the drawn direction may extend perpendicularly between two opposite edges of substrate 10 which define the longest dimension of substrate 10. Once the drawn direction has been determined substrate 10 is placed in position on station 20 such that drawn direction 110 is parallel with direction 204 or 63. Processor 140 is typically already programmed with the necessary layout information to fabricate target arrays 12. Processor 140 controls fabrication of each array by depositing one or more drops of each biopolymer onto a corresponding region (feature) on the substrate in the case of the deposition method, or by depositing biomonomer drops onto a region and sending the array to the flood station in the case of the in situ method, so as to fabricate the array (400). During operation, drops are deposited onto surface 11a from head 210 while moving head 210 along one of the rows parallel with drawn direction 110. This is repeated multiple times, each time at another one of the rows parallel with the drawn direction, until each array 12 is formed with linear rows of features 16 oriented parallel to drawn direction 110. During or following array fabrication, arrays may be inspected for quality control (“QC”), for example for information on missing features, misplaced features, features of incorrect dimensions, or other physical characteristics, in a manner as described in U.S. patent application Ser. No. 09/302898 for “Polynucleotide Array Fabrication” filed Apr. 30, 1999 by Caren et al., and application Ser. No. 09/419447 for “Biopolymer Array Inspection” filed Oct. 15, 1999 by Fisher, both incorporated herein by reference.

For each fabricated array 12, processor 140 will generate a corresponding unique identifier and will save this in memory 141 in association with the following: target array layout information (including the location and identity of biopolymers at each feature); quality control data; and an identification of the drawn direction 110. As previously mentioned, the form of the identification on drawn direction may simply be an instruction to a scanner to scan in an instructed direction, which is in fact the drawn direction 110. Either before array fabrication on substrate 10 has been commenced, or after it has been completed (or even during array fabrication), substrate 10 may be sent to writer 150 which, under control of processor 140, writes the identifier for each array 12 in the form of bar codes 356 onto substrate 10 each in association with its corresponding array (by being physically close to it in the manner shown in FIG. 1). The substrate 10 is then sent to a cutter 152 wherein portions of substrate 10 carrying an individual array 12 and its associated identifier 356 are separated from the remainder of substrate 10, to provide multiple array units 15. The array unit 15 is placed in package 340 along with a storage medium 324b (if used). Storage medium 324b (if used) may carry feature characteristic data, and the identification of the drawn direction 110, both in association with the identifier for that same array unit 15 (and possibly for other array units 15 which are to be sent to the same remote customer location). Package 340 is then shipped to a remote user station.

The identification of the drawn direction (the direction of rows of features) and the feature characteristic data 440 may both be associated with the array and forwarded to a remote location (typically a user station) using the same or different technique. For example, either or both may be forwarded to a remote user by saving on portable storage medium 324b in association with the corresponding array identifier, and shipping storage medium 324b (for example, in the same package 340 as the array unit 15) to the remote location, as already mentioned. Alternatively, the array unit 15 (with applied identifier 356) can be forwarded itself to the remote user such as by shipping. In this case, either or both of the information on the drawn direction and the feature characteristic data 440 may be separately forwarded by communication to a remote user station (specifically, to scanner 160) over channel 180 in response to a received communication from the remote station of the corresponding array identifier (as read by reader 163). An identification of the features in the array to which any data pertains, may be included as a part of the feature characteristic data. Note that the feature characteristic data may only be for a sub-set of features on a given array.

The above sequence can be repeated at the fabrication station as desired for multiple substrates 10 in turn. As mentioned above, the fabrication station may act as a central fabrication station for each of multiple remote user stations, in the same manner as described above. Whether or not the fabrication station acts as a central fabrication station, it can optionally maintain a database of the feature characteristic data and identification of the drawn direction for arrays, each in association with the corresponding array identifier. However, such database can be maintained at another location and may be dispensed with in the case where the identification of a direction on the array and the feature characteristic data are shipped on portable storage media (such as medium 324b) to the end users.

At the user station of FIG. 9, the resulting package 340 is then received from the remote fabrication station. A sample, for example a test sample, is exposed to the array 12 on the array unit 15 received in package 340. Following hybridization and washing in a known manner, the array unit 15 is then inserted into holder 161 in scanner 160 for reading of the array (such as information representing the fluorescence pattern on the array 12). The drawn direction is then determined in any of the manners previously described. For example, the array identifier may also be machine read by the reader 163 in scanner 160 reading the bar code 356 present on the array substrate 10 in association with the corresponding array 12, while the array unit 15 is still positioned in retained in holder 161. Using the read identifier 356, processor 162 may then retrieve the identification of the drawn direction and feature characteristic data for the array from either from portable storage medium 324b or from the database of such information in memory 141. The retrieval from memory 141 may be performed by communicating the array identifier to processor 140 through communication module 164 and communication channel 180, and receiving in response the identification and feature characteristic data for that array from memory 141. In the latter situation, processor 162 may obtain the communication address of communication module 144 by which to access memory 141 (or the address of another database carrying the identity map and associated identifier of array 12), from a communication address in identifier 356 or by accessing a database of manufacturer's communication addresses based on the read array identifier (either from a local memory or by communication with a remote database).

The array in array unit 15, while still positioned in holder 161, may be read to obtain read results. Processor 162 may cause the array to be read by scanning the illuminating beam sequentially along multiple rows of features 16 of the array and parallel to the drawn direction (typically in a line by line rectangular or zigzag manner already described). As mentioned, surface 11a will typically have a higher height uniformity in drawn direction 110 than in second direction 112. In this situation, focus will not have to be changed as frequently as would be the case if the illuminating beam was scanned sequentially along multiple columns (which extend in the second direction). The data obtained from reading may be processed (which term includes interpretation of data), using the retrieved feature characteristic data. For example, if the feature characteristic indicates a particular feature is missing or severely defective then the scanner may simply avoid reading such a feature at all. Alternatively, the read data from such a feature may simply be deleted or ignored in any subsequent processing, or processed results flagged as possibly being in error due to that defective feature. Results from the array reading can be also be processed by rejecting a reading for a feature which is below a predetermined threshold and/or forming conclusions based on the pattern read from the array (such as whether or not a particular target sequence may have been present in the sample). The results of the reading (processed or not) can be forwarded (such as by communication) to be received at a remote location for further evaluation and/or processing, or use, using communication channel 180 or reader/writer 186 and medium 190. This data may be transmitted by others as required to reach the remote location, or re-transmitted to elsewhere as desired.

In a variation of the above, it is possible that each array 12 and its substrate 10 may be contained with a suitable housing. Such a housing may include a closed chamber accessible through one or more ports normally closed by septa, which carries the substrate 10. In this case, the identifier 356 for each array may be applied to the housing.

Modifications in the particular embodiments described above are, of course, possible. For example, where a pattern of arrays is desired, any of a variety of geometries may be constructed other than the organized rows and columns of arrays 12 of FIG. 1. For example, arrays 12 can be arranged in a series of curvilinear rows across the substrate surface (for example, a series of concentric circles or semi-circles of spots), and the like. Similarly, the pattern of features 16 may be varied from the organized rows and columns in FIG. 2 to include, for example, a series of curvilinear rows across the substrate surface(for example, a series of concentric circles or semi-circles of spots), and the like. Even irregular arrangements of the arrays or the regions within them can be used provided the locations of features of identified biopolymers are known (and assuming a first direction can still be identified). Further, the identifier shipped to a remote user with the array need not be on the array substrate or housing provided it is in some manner associated with the corresponding array when shipped to the user. For example, the identifier could be only on portable storage medium 324b or a paper or other printed medium which is associated with the corresponding array such as by being physically associated with it in the same package 340.

The present methods and apparatus may be used to deposit biopolymers or other moieties on surfaces of any of a variety of different substrates, including both flexible and rigid substrates. Thus, in any of the above described methods “biopolymer” or “biopolymers” could more broadly be replaced with chemical or other “moiety” or “moieties”. Preferred materials for the substrate provide physical support for the deposited material and endure the conditions of the deposition process and of any subsequent treatment or handling or processing that may be encountered in the use of the particular array. The array substrate may take any of a variety of configurations ranging from simple to complex. Thus, the substrate could have generally planar form, as for example a slide or plate configuration, such as a rectangular or square or disc. In many embodiments, the substrate will be shaped generally as a rectangular solid, having a length in the range about 4 mm to 200 mm, usually about 4 mm to 150 mm, more usually about 4 mm to 125 mm; a width in the range about 4 mm to 200 mm, usually about 4 mm to 120 mm and more usually about 4 mm to 80 mm; and a thickness in the range about 0.01 mm to 5.0 mm, usually from about 0.1 mm to 2 mm and more usually from about 0.2 to 1 mm. However, larger substrates can be used, particularly when such are cut after fabrication into smaller size substrates carrying a smaller total number of arrays 12. Substrates of other configurations and equivalent areas can be chosen. The configuration of the array may be selected according to manufacturing, handling, and use considerations.

The substrates may be fabricated from any of a variety of materials. In certain embodiments, such as for example where production of binding pair arrays for use in research and related applications is desired, the materials from which the substrate may be fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, may be particularly useful in this embodiment. For rigid substrates, specific materials of interest include: glass; fused silica, silicon, plastics (for example, polytetrafluoroethylene, polypropylene, polystyrene, polycarbonate, and blends thereof, and the like); metals (for example, gold, platinum, and the like).

The substrate surface onto which the polynucleotide compositions or other moieties is deposited may be porous or non-porous, smooth or substantially planar, or have irregularities, such as depressions or elevations. The surface may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto (for example, conjugated),

Various further modifications to the particular embodiments described above are, of course, possible. Accordingly, the present invention is not limited to the particular embodiments described in detail above. greater variance in height has a lower height uniformity than a surface with a lower variance in height. “Variance” and “variation” in substrate surface height are used synonymously, and refer to the number of changes in height each greater than a predetermined value, along a predetermined path. An alternative measure of variance in substrate surface height, is the normal statistical quantity, which is the square of the standard deviation of height along the predetermined path. The height may be measured at two, four, ten or more points with reference to a line or plane extending along the path between opposite edges of the substrate surface. Thus, a simple way of measuring such variance is to measure the thickness of the substrate along the path. A “line” in reference to a series of features refers to those features which are laid out on a path extending in a same direction. A line may be a curved line (having one or more curvatures) or it may be a straight line A “row” of features of an array is a line of features which is most closely aligned with the first or drawn direction, than any other line of features. A “column” of features refers to a line of features which crosses a row, and is typically orthogonal to a row. By “more closely aligned” is meant having a lowest variance in distance or, in the case of straight lines of features and straight line first direction, the line with the least angle with the first or drawn direction. By “oriented adjacent” with reference to straight lines means less than 45 degrees (and may be less than 20, 10 or 5 degrees). By “drawn” direction in reference to a substrate, refers to the direction in which the substrate may have been pulled or pushed during a conventional drawing operation (including being pulled through a slot or over a roller, or being extruded through an opening). Drawn directions are therefore straight line directions while first and second directions may be straight line directions but not necessarily so.

It will also be appreciated that throughout the present application, that words such as “top”, “upper”, and “lower” are used in a relative sense only. “Fluid” is used herein to reference a liquid. A “set” or a “sub-set” may have one or more members (for example, one or more droplets). A “processor” includes any one or more electrical and/or optical processors which can execute all the steps required of it, or any hardware or software combination which will perform those or equivalent steps, such as one or more general purpose digital microprocessors suitably programmed from a computer readable medium carrying necessary program code. Any “memory” includes any suitable device or combination of devices in which a processor can store and/or retrieve data as required, such as magnetic, optical, or solid state storage devices (including magnetic or optical disks or tape or RAM, or any other suitable device or combination of them, either fixed or portable). Steps recited in a particular order in relation to any method can be performed in that order or changed to any order which is logically possible. Reference to a singular item, includes the possibility that there are plural of the same items present. All patents and other cited references are incorporated into this application by reference.

Referring first to FIGS. 1-5, methods and apparatus of the present invention may generate or use a contiguous planar substrate 10 (such as a glass substrate) carrying one or more arrays 12 disposed across a front surface 11a of substrate 10 and separated by interarray areas 13. A back surface 11b of substrate 10 does not carry any arrays 12. Substrate 10 illustrated, is rectangular in shape and was manufactured by drawing in a direction 110, with a direction 112 extending across drawn direction 110 (specifically, direction 112 is orthogonal to drawn direction 110). Drawn direction 110 will typically be what is referenced as a “first direction” in the present application, while direction 112 will typically be a “second direction” as referenced herein. Substrate 10 should preferably have relatively low or no substantial amounts of any fluorescent components which may interfere with detection of fluorescence during reading of an array 12. Thus, typical float glass (typically formed by floating molten glass on a layer of molten tin) is not as desirable for use as an array substrate since it contains tin ions which may fluoresce. A suitable drawn glass is a downwardly drawn glass such as available from Schott Glass Technologies, Duryea, PA, or Schott Glass, Germany. The arrays on substrate 10 can be designed for testing against any type of sample, whether a trial sample, reference sample, a combination of them, or a known mixture of polynucleotides (in which latter case the arrays may be composed of features carrying unknown sequences to be evaluated). Each array 12 has associated with it a unique identifier in the form of a bar code 356 described below. By “unique” in this sense does not mean the identifier is absolutely unique, but it is sufficiently long so as unlikely to be confused with another identifier on another array (and is preferably unique as to a particular central fabrication station on a given communication channel). While ten arrays 12 are shown in FIG. 1 and the different embodiments described below may use a substrate with only one array 12 on it, it will be understood that substrate 10 and the embodiments to be used with it may have any number of desired arrays 12. Similarly, substrate 10 may be of any shape, and any apparatus used with it adapted accordingly. Depending upon intended use, any or all of arrays 12 may be the same or different from one another and each will contain multiple spots or features 16 of biopolymers such as polynucleotides.

Array 12 may contain from more than ten, more than one hundred, more than one thousand or ten thousand features, or even more than one hundred thousand features. Features 16 as shown are arranged in a pattern of orthogonal straight line rows and straight line columns, with rows laid out parallel to a drawn direction 110 of substrate 10 and columns laid out parallel to a direction 112 orthogonal to the drawn direction 110. All of the features 16 may be different, or some or all could be the same. In the embodiment illustrated, there are interfeature areas 17 between features, which do not carry any polynucleotide. It will be appreciated though, that the interfeature areas 17 could be of various sizes and configurations. However, there need not be any space separating arrays 12 from one another, nor features 16 within an array from one another. However, in the case where arrays 12 are formed by the deposition method as described above, such inter-array and inter-feature areas 17 will typically be present. Each feature carries a predetermined polynucleotide (which includes the possibility of mixtures of polynucleotides). As per usual, A, C, G, T represent the usual nucleotides. It will be understood that there may be a linker molecule (not shown) of any known types between the front surface 11a and the first nucleotide.

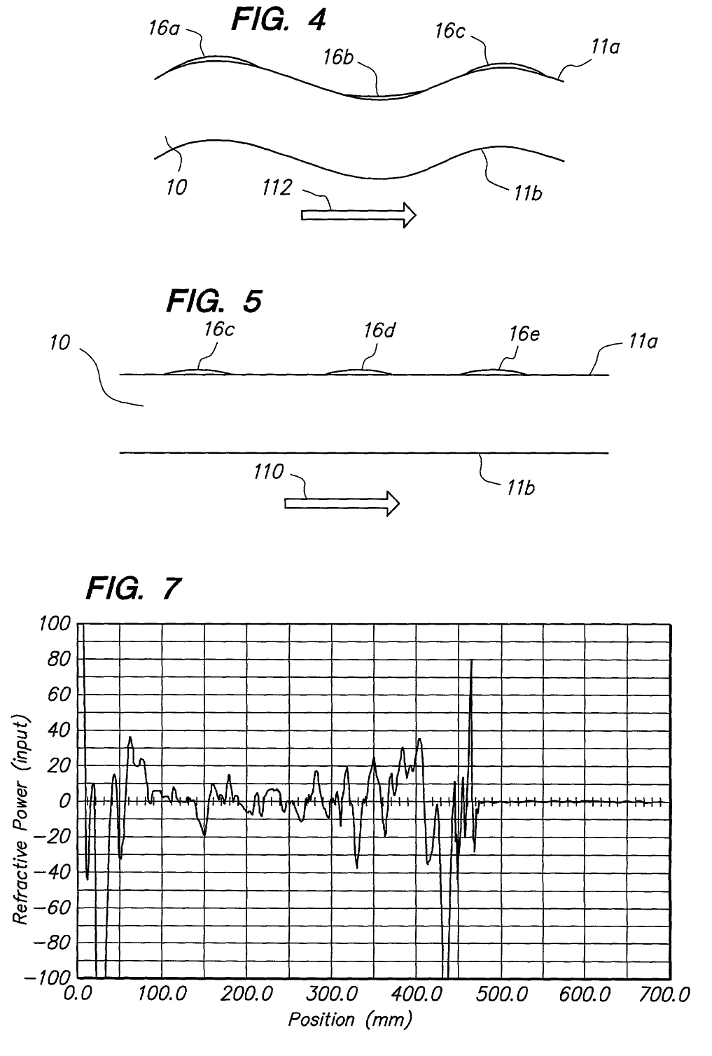

FIGS. 2 and 3 are enlarged views illustrating features 16 of an array 12 in FIG. 1. Ideally, the features 16 would be uniform in shape, size and composition, and the features regularly spaced. In practice, such an ideal result is difficult to obtain particularly when there are thickness variations in substrate 10 or other height irregularities in upper surface 11a. In particular, referring to FIGS. 3-5, substrate 10 may be a drawn glass, having been drawn in the drawn direction 110 over a roller. As a result, while there may be relatively little variance in height of upper surface 11a in the drawn direction 110, there may be substantially more variance in the direction 112 as illustrated in FIG. 3 (somewhat exaggerated and not to scale, for clarity). Note the variation in height of each of the features 16a, 16b, 16c in a row of features extending parallel to second direction 112. In the situation in FIG. 3, lower surface 11b is planar in direction 112 but this need not be so. For example, as illustrated for a substrate in FIG. 4, both upper and lower surfaces 11a, 11b are parallel with both having a same variance in height. As can be seen from FIGS. 4 and 5, there is a greater variance in height between features 16a, 16b, 16c in a row of features (which extends in the drawn direction 110) than between features 16c, 16d, 16e in a column of features (which extends in second direction 112). The identity of the directions 110 and 112 can be determined by an array fabricator in any number of ways. For example, a thickness of substrate 10 can be measured at different positions on substrate 10. In the case where substrate 10 is rectangular, this can be done by measuring the thickness at pairs of opposing ends of the substrate. Further additional measurements can be made to obtain a more accurate result. Alternatively, the determining can be performed based on an identification of the first or drawn direction received at a fabrication station from a remote location, which identification is associated with the substrate. Such identification may be in writing, saved on a portable memory, or communicated electronically (for example, from a remote memory over a network such as the Internet) in response to a received corresponding identification of the substrate (such as an identifier on the substrate, such as a bar code, or a description of the substrate). The association with the substrate may be accomplished, for example, by packaging a medium carrying the identification with the substrate, or the association may result at the fabrication station as a result of communicating the identification there in response to the received identifier or description. The identification may, for example, identify the drawn direction by any means such as by reference to one or more fiducial marks on the substrate (for example, the line between two identified marks is the drawn direction) or may identify the drawn direction by reference to a shape characteristic of substrate 10 (for example, the drawn direction of a rectangular substrate may be identified as the longest direction of the substrate). The identification may be received from a same or different location (such as a same remote location) from which substrate 10 was received.

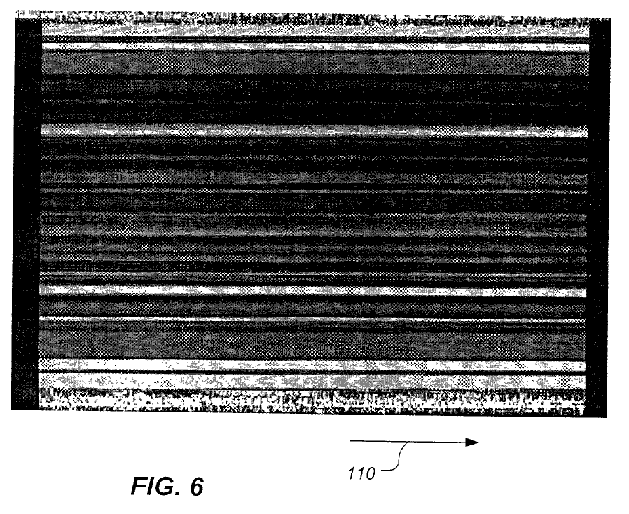

FIGS. 6 and 7 illustrate refractive power measurement (in millidiopters) of an actual specimen of drawn glass about 1 mm in thickness as might be used as a substrate on which an array is fabricated by a method of the present invention. In FIG. 6 lines of equal color represent the same refractive power measurement and therefore provides a measurement of uniformity of the glass thickness. Note that the drawn direction in FIG. 6 is along the horizontal direction as viewed in the FIG. 6. FIG. 7 shows the refractive power measurement (vertical axis in FIG. 7) taken at 90 degrees across the drawn direction of the same piece of glass of FIG. 6, versus the position across the width of the glass in millimeters (horizontal axis in FIG. 7). As can be seen from FIGS. 6 and 7 then, the glass thickness of the glass specimen is fairly uniform in the drawn direction, but varies substantially more across the drawn direction.

Referring now to FIG. 8, an apparatus which can execute a method of the present invention, will now be described. The apparatus of FIG. 8 is a central fabrication station which includes a substrate station 20 on which can be mounted a substrate 10. Pins or similar means (not shown) can be provided on substrate station 20 by which to approximately align substrate 10 to a nominal position thereon. Substrate station 20 can include a vacuum chuck connected to a suitable vacuum source (not shown) to retain a substrate 10 without exerting too much pressure thereon, since substrate 14 is often made of glass.

A dispensing head 210 is retained by a head retainer 208. The positioning system includes a carriage 62 connected to a first transporter 60 controlled by processor 140 through line 66, and a second transporter 100 controlled by processor 140 through line 106. Transporter 60 and carriage 62 are used execute one axis positioning of station 20 (and hence mounted substrate 10) facing the dispensing head 210, by moving it in the direction of arrow 63, while transporter 100 is used to provide adjustment of the position of head retainer 208 (and hence head 210) in a direction of axis 204. In this manner, head 210 can be scanned line by line, by scanning along a line over substrate 10 in the direction of axis 204 using transporter 100, while line by line movement of substrate 10 in a direction of axis 63 is provided by transporter 60. In the case where arrays 12 are to be fabricated by the deposition method, transporter 60 can also move a load station (not shown) beneath head 210 such that polynucleotides or other biopolymers obtained from different vessels from a customer, can be loaded into head 210. Such a load station and method of use is described in detail in U.S. patent application Ser. No. 09/183,604 for “Method And Apparatus For Liquid Transfer” filed Oct. 30, 1998 by Tella et al, incorporated herein by reference. In the case where arrays 12 are to be fabricated by the in situ method, supplies of suitable reagents can be provided in fluid communication with head 210, and a flood station can be provided for steps in the process in which all features to be formed are exposed to the same solution. Such features are described in more detail in U.S. patent application Ser. No. 09/356249 for “Biopolymer Arrays And Their Fabrication” filed by Perbost on Jul. 16, 1999, incorporated herein by reference. Head 210 may also optionally be moved in a vertical direction 202 toward and away from substrate station 20, by another suitable transporter (not shown). It will be appreciated that other scanning configurations could be used. It will also be appreciated that both transporters 60 and 100, or either one of them, with suitable construction, could be used to perform the foregoing scanning of head 210 with respect to substrate 10. Thus, when any reference is made in the present application to “positioning” or “moving” one element (such as head 210) in relation to another element (such as one of the stations 20 or substrate 10), it is to be understood that any required positioning or moving can be accomplished by moving either element or a combination of both of them. The head 210, the positioning system, and processor 140 together act as the deposition system of the apparatus. An encoder 30 communicates with processor 140 to provide data on the exact location of substrate station 20 (and hence substrate 10 if positioned correctly on substrate station 20), while encoder 34 provides data on the exact location of holder 208 (and hence head 210 if positioned correctly on holder 208). Any suitable encoder, such as an optical encoder, may be used which provides data on linear position.

Processor 140 also has access through a communication module 144 to a communication channel 180 to communicate with one or more remote stations, such as locations at which arrays 12 are read. Communication channel 180 may, for example, be a Wide Area Network (“WAN”), telephone network, satellite network, or any other suitable communication channel. Communication module 144 may be any module suitable for the type of communication channel used, such as a computer network card, a computer fax card or machine, or a telephone or satellite modem.

Head 210 may have multiple pulse jets, such as piezoelectric or thermoelectric type pulse jets as may be commonly used in an ink jet type of printer and may, for example, include multiple chambers each communicating with a corresponding set of multiple drop dispensing orifices and multiple ejectors which are positioned in the chambers opposite respective orifices. Each ejector is in the form of an electrical resistor operating as a heating element under control of processor 140 (although piezoelectric elements could be used instead). Each orifice with its associated ejector and portion of the chamber, defines a corresponding pulse jet. It will be appreciated that head 210 could, for example, have more or less pulse jets as desired (for example, at least ten or at least one hundred pulse jets). Application of a single electric pulse to an ejector will cause a droplet to be dispensed from a corresponding orifice. Certain elements of the head 210 can be adapted from parts of a commercially available thermal inkjet print head device available from Hewlett-Packard Co. as part no. HP51645A. A suitable head construction is described in U.S. patent application Ser. No. 09/150,507 filed Sep. 9, 1998 by Caren et al. for “Method And Multiple Reservoir Apparatus For Fabrication Of Biomolecular Arrays”, incorporated herein by reference. Alternatively, multiple heads could be used instead of a single head 210, each being similar in construction to head 210 and being movable in unison by the same transporter or being provided with respective transporters under control of processor 140 for independent movement.

As is well known in the ink jet print art, the amount of fluid that is expelled in a single activation event of a pulse jet, can be controlled by changing one or more of a

What is claimed is:

1. A method of fabricating an array of multiple features of different chemical moieties on a substrate surface, comprising:
(a) determining an identity of a first direction across the substrate surface along which the substrate surface has a higher height uniformity than along a second direction across the substrate; and
(b) placing the chemical moieties on the substrate so as to provide features thereon along rows more closely aligned with the first direction than the second direction.

2. A method according to claim 1 wherein the different chemical moieties are biopolymers.

3. A method according to claim 1 wherein the determining comprises measuring the thickness of the substrate at different positions.

4. A method of fabricating an array of multiple features of different chemical moieties on a substrate surface, comprising:
(a) receiving the substrate from a remote location;
(a) receiving from a remote location in association with the substrate, an identification of a first direction across the substrate surface along which the substrate surface has a higher height uniformity than along a second direction across the substrate;
(b) placing the chemical moieties on the substrate so as to provide features thereon along rows more closely aligned with the first direction than the second direction.

5. A method according to claim 1 additionally comprising associating with the array an identification as to the direction of the rows and forwarding the array and associated identification to a remote location.

6. A method according to claim 5 wherein the forwarding of the identification comprises applying an identifier on the substrate or a housing for the substrate, and saving the identification in a memory in association with the identifier.

7. A method according to claim 5 wherein the identification comprises reference to a shape characteristic of the substrate or a housing for the substrate.

8. A method according to claim 1 wherein the substrate is rectangular and the first and second directions extend perpendicularly between respective sets of opposite edges of the substrate.

9. A method according to claim 1 wherein the rows are parallel with the first direction.

10. A method of fabricating an array of multiple features of different chemical moieties on a drawn substrate, comprising:
(a) determining an identity of a drawn direction of the substrate;
(b) placing the chemical moieties on the substrate so as to provide features thereon along linear rows oriented adjacent the drawn direction.

11. A method according to claim 10 wherein the determining comprises measuring the thickness of the substrate.

12. A method of fabricating an array of multiple features of different chemical moieties on a surface of a drawn substrate, comprising:
(a) receiving the drawn substrate from a remote location;
(a) receiving from a remote location in association with the substrate, an identification of the drawn direction; and
(b) placing the chemical moieties on the substrate surface so as to provide features thereon along linear rows oriented parallel to the drawn direction.

13. A method according to claim 10 wherein the rows are parallel with the drawn direction.

14. A method according to claim 11 wherein the substrate is rectangular and the drawn direction extends perpendicular to and between opposite edges of the substrate.

15. A method according to claim 10 additionally comprising associating with the array an identification as to the direction of the rows and forwarding the array and associated identification to a remote location.

16. A method according to claim 15 wherein the forwarding of the identification comprises applying an identifier on the substrate or a housing for the substrate, and saving the identification in a memory in association with the identifier.

17. A method according to claim 15 wherein the identification comprises reference to shape characteristic of the substrate or a housing for the substrate.

18. A method of fabricating an array of multiple linear rows of features of different chemical moieties on a surface of a drawn rectangular substrate, comprising:
(a) receiving the drawn substrate from a remote location;
(a) determining an identity of a drawn direction of the substrate; and
(b) placing the chemical moieties on the substrate surface so as to provide features thereon along linear rows oriented parallel to the drawn direction, wherein the placing comprises:
(i) depositing drops onto the surface from a drop deposition head while moving the head along one of the rows parallel with the drawn direction;
(ii) repeating step (i) multiple times, each time at another one of the rows parallel with the drawn direction, so as to form the array.

19. A method according to claim 18 wherein the determining comprises receiving an identification of the drawn direction from a remote location in association with the substrate.

20. A method of reading an array of multiple features of different chemical moieties on a substrate surface, the array having rows of features, comprising:
(a) determining an identity of a first direction across the substrate surface along which the substrate thickness has a higher height uniformity than along a second direction across the substrate; and
(b) repeatedly scanning an illuminating beam across features in parallel paths which are more closely aligned with the first direction than the second direction.

21. A method according to claim 20 wherein the determining is based on an identifier carried on the substrate or a housing for the substrate.

22. A method according to claim 21 wherein the determining is performed by retrieving an identification of the first direction from the identifier.

23. A method according to claim 21 wherein the determining is performed by retrieving an identification of the first direction from a memory in response to providing the identifier.

24. A method of reading an array of multiple features of different chemical moieties arranged on a surface of a drawn rectangular substrate in linear rows extending parallel to a direction in which the substrate was drawn, comprising:
(a) determining an identity of a drawn direction of the substrate;
(b) scanning an illuminating beam sequentially along multiple rows of the array and parallel to the drawn direction.

25. A method according to claim 20 wherein the determining is based on an identifier on the substrate or a housing for the substrate.

26. A method according to claim 20 wherein the determining comprises measuring the thickness of the substrate at different positions.
